(19) United States
(12) Patent Application Publication
(10) Pub. No.: US 2012/0253454 A1
(43) Pub. Date: Oct. 4, 2012

(54) STENT DESIGNS HAVING ENHANCED RADIOPACITY

(76) Inventor: Kieran Costello, Co. Clare (IE)

(21) Appl. No.: 13/430,846

(22) Filed: Mar. 27, 2012

Related U.S. Application Data
(60) Provisional application No. 61/470,196, filed on Mar. 31, 2011.

(51) Int. Cl. A61F 2/84

(52) U.S. Cl. 623/1.34

(57) ABSTRACT

The present embodiments provide stents for use in medical procedures. In one embodiment, a stent comprises a first flanged region and a body region. A first diameter of the first flanged region is greater than a second diameter of the body region when the stent is in an expanded deployed state. A proximal junction is formed between the first flanged region and the body region. The proximal junction comprises at least one strut extending from the distal end of the first flanged region in a distal direction towards the proximal end of the body region. A strut at the proximal end of the body region is disposed around at least a portion of the strut of the proximal junction. The overlap between the strut at the proximal end of the body region with the strut of the proximal junction causes an increased radiopaque effect at the proximal junction.

### PRIORITY CLAIM

This invention claims the benefit of priority of U.S. Provisional Application Ser. No. 61/470,196, entitled “Stent Designs Having Enhanced Radiopacity,” filed Mar. 31, 2011, the disclosure of which is hereby incorporated by reference in its entirety.

### BACKGROUND

The present embodiments relate generally to medical devices, and more particularly, to stent designs having enhanced radiopacity.

Stents may be inserted into an anatomical vessel or duct to maintain or restore patency in a constricted passageway, or may be used for other purposes. Stents may be manufactured using materials such as plastic or metal, and may comprise a variety of configurations, for example, a wire-mesh, coil or helical shape, or a slotted tube configuration.

Stents may be self-expanding or balloon expandable, or combinations thereof. A self-expanding stent may be delivered to a target site in a compressed configuration and subsequently expanded by removing a delivery sheath. In such embodiments, the stent may comprise a shape-memory alloy such as nitinol that allows the stent to return to a predetermined configuration upon removal of the sheath. By contrast, a balloon expandable stent may be delivered using a balloon catheter. In such a procedure, the catheter may be inserted over a wire guide into a vessel or duct and advanced until the stent is aligned at the target site, and the stent then may be deployed by inflating the balloon to expand the stent diameter, whereby the stent engages and may slightly expand the lumen diameter of the vessel or duct.

When deploying a stent according to either self-expanding or balloon expandable techniques, it is important for a physician to clearly view the stent, or at least portions of the stent, using a suitable imaging modality, such as fluoroscopy. In particular, it may be desirable to view selected regions of a stent, such as the proximal end, the distal end, regions to be aligned with a stricture, and/or other pertinent areas during placement of the stent. For example, when implanting a stent across a stricture, it may be desirable or necessary to identify the boundaries of the stent portion to be disposed across the stricture versus other portions of the stent that are intended to be disposed proximal and distal to the stricture.

Various existing stents employ radiopaque markers, which may comprise a material such as tantalum, platinum, gold, or another imageable material, that is coupled to the stent in a region of interest. However, such radiopaque markers are generally limited in size based on the strut portion to which they are attached, and can therefore appear relatively small when viewed under fluoroscopy or other techniques.

Still other stents attempt to increase visibility during implantation by providing thicker wire cross-sections. However, increasing the wire thickness may reduce flexibility of the individual struts forming the stent, and may cause wires to straighten the lumen of the duct or vessel into which they are implanted, which can lead to patient discomfort and possible perforation of a passageway. Further, if such a stent is placed in a passageway such as the lower esophageal sphincter, the stent may exacerbate gastroesophageal reflux by not allowing the lower esophageal sphincter to close properly. In sum, providing thicker wire cross-sections and/or radiopaque markers are not always desirable solutions for enhanced visualization of selected regions of a stent.

### SUMMARY

The present embodiments provide stents for use in medical procedures. In one embodiment, a stent comprises a first flanged region and a body region. A first diameter of the first flanged region is greater than a second diameter of the body region when the stent is in an expanded deployed state. A proximal junction is formed between the first flanged region and the body region. The proximal junction comprises at least one strut extending from the distal end of the first flanged region in a distal direction towards the proximal end of the body region. A strut at the proximal end of the body region is disposed around at least a portion of the strut of the proximal junction. The overlap between the strut at the proximal end of the body region with the strut of the proximal junction causes an increased radiopaque effect at the proximal junction.

In one embodiment, the strut of the proximal junction comprises first and second segments that extend distally from the first flanged region and converge at an apex. The strut at the proximal end of the body region may be disposed around the apex. Optionally, at least one separate loop member may encircle a zone in which the strut at the proximal end of the body region is disposed around the apex.

In one embodiment, the first and second segments of the strut of the proximal junction may form an integral loop member at the apex, and the strut at the proximal end of the body region is disposed through the integral loop member. In an alternative embodiment, an integral loop member is not formed, but rather an external loop member is coupled to the strut of the proximal junction at the apex. In the latter embodiment, the strut at the proximal end of the body region is disposed through the external loop member.

In a further alternative embodiment, the strut of the proximal junction comprises first and second segments that extend distally from the first flanged region and converge at an apex, wherein the apex is folded over in a proximal direction to form a generally W-shape in the strut of the proximal junction. The strut at the proximal end of the body region is disposed around the W-shape formed in the strut of the proximal junction. For example, the strut at the proximal end of the body region may be disposed in front of the W-shape at least two times, and may be disposed behind the W-shape at least two times.

Advantageously, in all of the embodiments, the proximal junction comprises a shape that enhances radiopacity when viewed using a suitable imaging modality, without the need to provide wider strut cross-sections or separate radiopaque markers. The enhanced radiopacity may allow a physician to readily identify the proximal junction during placement of the stent, which may be beneficial particularly when placing only the main body region within a target area such as a stricture.

Other systems, methods, features and advantages of the invention will be, or will become, apparent to one with skill in the art upon examination of the following figures and detailed description. It is intended that all such additional systems, methods, features and advantages be within the scope of the invention, and be encompassed by the following claims.

### BRIEF DESCRIPTION OF THE DRAWINGS

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is an elevated perspective view of a first embodiment of a stent.

FIG. 2 is an elevated perspective view illustrating features of a junction of the stent of FIG. 1.

FIGS. 3-6 are elevated perspective views of alternative junctions of the stent of FIG. 1.

FIGS. 7A-7B are, respectively, fluoroscopic images showing features of the stent of FIG. 1 and an alternative stent having tantalum marker bands.

### DETAILED DESCRIPTION OF THE PREFERRED EMBODIMENTS

In the present application, the term “proximal” refers to direction that is generally towards a physician during a medical procedure, while the term “distal” refers to a direction that is generally towards a target site within a patent's anatomy during a medical procedure.

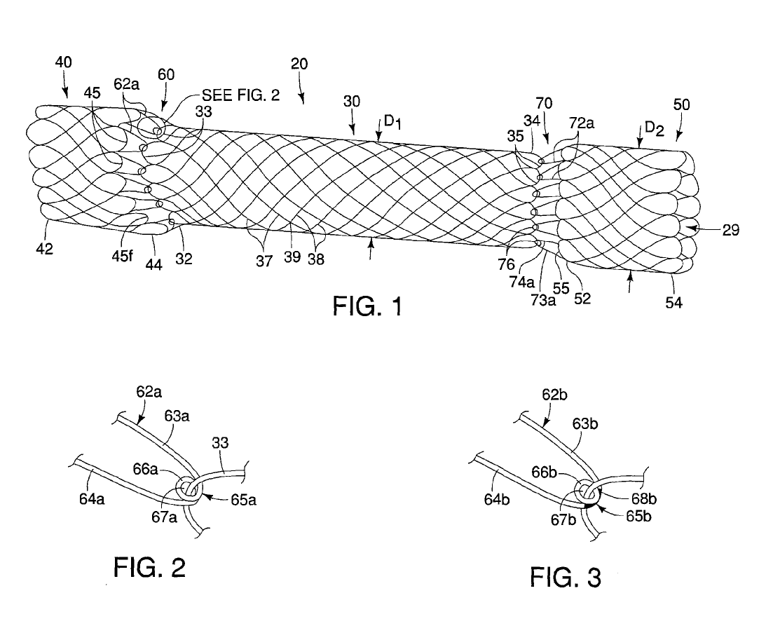

Referring now to FIG. 1, a first embodiment of a stent 20 according to the present embodiments is shown. The stent 20 may comprise any suitable shape and may be made from any suitable material, as long as consistent with the principles herein, as explained further below.

In the embodiment of FIG. 1, the stent 20 comprises a body region 30, as well as first and second flanged regions 40 and 50. The body region 30 has proximal and distal ends 32 and 34, respectively. The first flanged region 40 has proximal and distal ends 42 and 44, respectively, while the second flanged region 50 has proximal and distal ends 52 and 54, respectively, as shown in FIG. 1.

The stent 20 has a delivery state that is suitable for insertion into a target duct or vessel of a patient, and an expanded deployed state as shown in FIG. 1. A lumen 29 is formed between the first flanged region 40, the body region 30, and the second flanged region 50 to permit fluid flow throughout the length of the stent 20. In the expanded deployed state, the stent 20 has structural characteristics that are suitable for a particular application, such as radial force requirements to maintain patency within a vessel or duct.

The stent 20 further comprises a proximal junction 60 and a distal junction 70. The proximal junction 60 couples the distal end 44 of the first flanged region 40 to the proximal end 32 of the body region 30, as shown in FIG. 1. The distal junction 60 couples the proximal end 52 of the second flanged region 50 to the distal end 34 of the body region 30.

In the expanded deployed state of FIG. 1, the body region 30 comprises an outer diameter D₁, while the first and second flanged regions 40 and 50 each comprise outer diameters D₂, wherein the outer diameter D₂ is greater than the outer diameter D₁. In this manner, the proximal junction 60 serves as a tapered region transitioning from the outer diameter D₂ of the first flanged region 40 to the outer diameter D₁ of the body region 30, while the distal junction 70 serves as a tapered region transitioning from the outer diameter D₂ of the second flanged region 50 to the outer diameter D₁ of the body region 30, as shown in FIG. 1.

In accordance with one aspect, the proximal and distal junctions 60 and 70 each comprise shapes that enhance radiopacity when viewed using a suitable imaging modality, as explained in further detail below. The enhanced radiopacity advantageously may allow a physician to readily identify the proximal and distal junctions 60 and 70 during placement of the stent 20, which may be beneficial particularly when placing only the body region 30 within a region such as a stricture.

In the embodiment of FIGS. 1-2, the proximal junction 60 comprises a series of struts 62a that are coupled to corresponding struts 33 formed at the proximal end 32 of the body region 30. As best seen in FIG. 2, each strut 62a comprises first and second segments 63a and 64a, respectively, which converge at a distal apex 65a. In this embodiment, a loop member 66a is integrally formed by the first and second segments 63a and 64a coming together. The strut 33 at the proximal end 32 of the body region 30 is disposed through the loop member 66a of the proximal junction 60, as shown in FIGS. 1-2. In this manner, each of the struts 62a of the proximal junction 60 may be coupled to corresponding struts 33 of the body region 30.

In one embodiment, the struts 62a of the proximal junction 60 may be formed integrally with struts 45 at the distal end 44 of the first flanged region 40. In an alternative embodiment, the struts 62a may be formed as separate strut members that are coupled to the struts 45 using solder, a weld, an adhesive, a mechanical connection, and the like. It is not necessary that each strut 45 is coupled to a strut 62a. For example, one particular strut 45f of the first flanged region 40 lacks a direct coupling to the body region 30 via a strut 62a, as shown in FIG. 1.

The second flanged region 50 may be coupled to the body region 30 via the distal junction 70 in a similar manner. In particular, the distal junction 70 comprises a series of struts 72a having first and second segments 73a and 74a, and a loop 76 through which corresponding struts 35 formed at the distal end 34 of the body region 30 are disposed. The characteristics of the struts 72a of the distal junction 70 may be identical to the struts 62a of the proximal junction 60, as explained in FIG. 2. In alternative embodiments, the characteristics of the struts of the distal junction 70 may be formed in accordance with alternative struts 62b-62e of the proximal junction 60, as described in FIGS. 3-6 below, respectively.

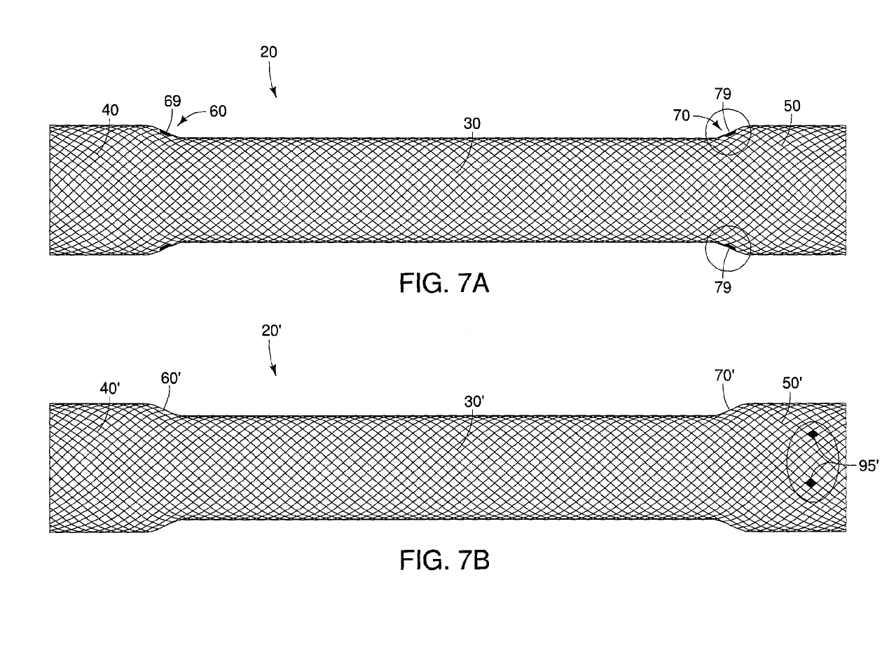

Referring to FIGS. 7A-7B, the advantages of applicants' stent structure are shown in connection with different prototypes viewed under fluoroscopy. FIG. 7A shows a prototype provided in accordance with the stent 20 under fluoroscopy. As can be seen, the combination of the struts 62a having first and second segments 63a and 64a, and a loop 66a through which the strut 33 is disposed, provides one or more localized, readily identifiable proximal markers 69 at the proximal junction 60. Similarly, the combination of the struts 72a having first and second segments 73a and 74a, and a loop 76 through which the strut 35 is disposed, provides one or more localized, readily identifiable distal markers 79 at the distal junction 70, as shown in FIG. 7A.

FIG. 7B shows an alternative stent 20′ that is structurally similar to the stent 20, having a main body 30′, first and second flanged regions 40′ and 50′, and proximal and distal junctions 60′ and 70′. A plurality of separate tantalum marker bands 95′ are disposed on the second flanged region 50′. As can been seen, the proximal and distal markers 69 and 79 formed at the junctions 60 and 70, respectively, of the stent 20 of FIG. 7A are as identifiable, if not more identifiable, under fluoroscopy than the plurality of separate tantalum marker bands 95′ of the stent 20′ of FIG. 7B.

Advantageously, the enhanced radiopacity may allow a physician to readily identify the proximal and distal junctions 60 and 70 during placement of the stent 20, which may be beneficial particularly when placing only the body region 30 within a target area such as a stricture. Moreover, the proximal and distal junctions 60 and 70 comprise shapes that enhance radiopacity when viewed using a suitable imaging modality, such as fluoroscopy, without the need to provide separate radiopaque markers that may be limited in size due to the diameter of the wire to which they are attached. Further, the proximal and distal junctions 60 and 70 comprise shapes that enhance radiopacity without the need to provide wider strut cross-sections that can add to a bulky delivery profile and have a tendency to straighten when implanted in a curved vessel or duct.

For example, in the embodiment of FIG. 1 and FIG. 7A, the outer diameter of the various wire segments forming the stent 20 may be between about 0.10 mm to about 0.30 mm. Advantageously, a stent formed from wires of such relatively small outer diameter may better conform to anatomy, reduce the likelihood of straightening a duct or vessel, reduce potential perforations and patient discomfort, while achieving the imaging benefits described and shown in FIG. 7A due to the specific structural arrangements provided.

Referring now to FIGS. 3-6, various alternative couplings along the proximal junction 60 are shown to provide enhanced radiopacity between the first flanged region 40 and the body region 30. In FIG. 3, the proximal junction 60 comprises a series of struts 62b that are coupled to corresponding struts 33 formed at the proximal end 32 of the body region 30. Each strut 62b comprises first and second segments 63b and 64b that converge at a distal apex 65b. In this embodiment, a loop member 66b is externally formed and then secured to the struts 62b at an attachment point 68b in the vicinity of the distal apex 65b, as shown in FIG. 3. For example, the loop member 66b may be secured at the attachment point 68b using a solder, weld, adhesive or mechanical coupling device. The strut 33 at the proximal end 32 of the body region 30 is disposed through the loop member 66b. In view of the localized imageable material provided by the loop member 66b, in addition to the strut segment 62b and the strut 33, the coupling arrangement shown in FIG. 3 is intended to provide enhanced localized radiopacity along the proximal junction 60 at a location between the first flanged region 40 and the body region 30, in a manner similar to that described in FIGS. 1-2 above.

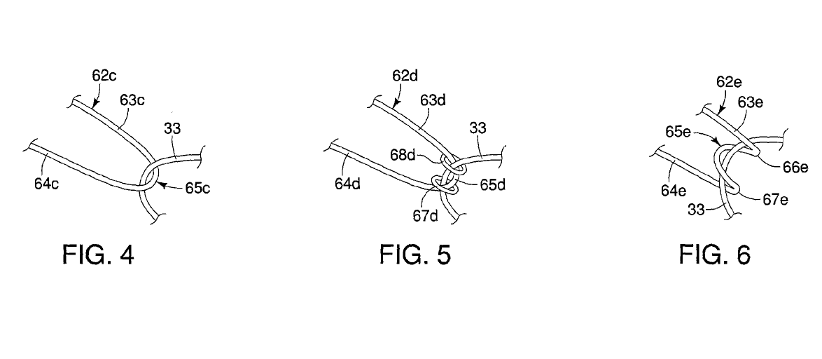

Referring to FIG. 4, the proximal junction 60 comprises a series of struts 62c that are coupled to the struts 33 formed at the proximal end 32 of the body region 30. Each strut 62c comprises first and second segments 63c and 64c that converge at a distal apex 65c. The strut 33 at the proximal end 32 of the body region 30 is disposed around the apex 65c. In this example, unlike FIGS. 2-3, a loop member is omitted. However, the coupling arrangement shown in FIG. 4, with the overlap of the strut 33 with the apex 65c, is expected to provide localized enhanced radiopacity along the proximal junction 60 at a location between the first flanged region 40 and the body region 30, in a manner similar to that described in FIGS. 1-2 above. Optionally, a frictional coating, such as silicone, may be provided on a portion of the struts 33 and/or the struts 62c to help reduce relative movement of the stent sections 30 and 40 in this particular embodiment.

Referring to FIG. 5, the proximal junction 60 comprises a series of struts 62d that are coupled to the struts 33 formed at the proximal end 32 of the body region 30. Each strut 62d comprises first and second segments 63d and 64d that converge at a distal apex 65d, like the embodiment of FIG. 4. The strut 33 at the proximal end 32 of the body region 30 is disposed around the apex 65d. In this example, unlike FIG. 4, one or more additional loop members, e.g., loop members 67d and 68d, are separately looped around a zone in which the strut 33 is disposed around the apex 65d. In view of the localized imageable material provided by the one or more additional loop members 67d and 68d, in addition to the strut segment 63d and 64d and the strut 33, the coupling arrangement of FIG. 5 is intended to provide enhanced radiopacity along the proximal junction 60 at a location between the first flanged region 40 and the body region 30, in a manner similar to that described in FIGS. 1-2 above.

Referring to FIG. 6, the proximal junction 60 comprises a series of struts 62e that are coupled to the struts 33 formed at the proximal end 32 of the body region 30. Each strut 62e comprises first and second segments 63e and 64e that converge at a distal apex 65e. In the embodiment of FIG. 6, the apex 65e is folded over in a proximal direction at bend locations 66e and 67e, thereby forming a generally “W-shape” in the strut 62e. The strut 33 at the proximal end 32 of the body region 30 is disposed around the strut 62e. In one example, the strut 33 may be disposed in front of the “W-shaped” bend of strut 62e two times, and behind the “W-shaped” bend two times, as shown in FIG. 6. In view of the localized imageable material provided by the “W-shaped” bend in the strut 62e, and the wire 33 passing through this region, the coupling arrangement of FIG. 6 is intended to provide enhanced radiopacity along the proximal junction 60 at a location between the first flanged region 40 and the body region 30.

In any of the embodiments above, the coupling features shown in FIGS. 2-6 may be reversed, such that loop members 66a, 66b, W-shaped strut 65e, and other features shown integral or coupled to the struts 62 of the proximal junction 60 may be instead provided as part of the struts 33 formed at the proximal end 32 of the body region 30. In such alternative embodiments, the struts 62 of the proximal junction 60 may take the generally arched shape of the struts 33, while the struts 33 have the features shown for the struts 62, yet the resulting coupling and functionality of the stent 20 is generally the same and its visibility is still enhanced.

Further, in any of the embodiments above, various types of stents may be used along the body region 30, as well as the first and second flanged regions 40 and 50. In the example of FIG. 1, the body region 30, as well as the first and second flanged regions 40 and 50, each comprise a braided stent that may comprise one or more wires that are formed into a desired braided pattern. For example, the body region 30 comprises a plurality of first wire segments 37 extending in a first direction and a plurality of second wire segments 38 extending in a second direction. The plurality of first wire segments 37 intersect the plurality of second wire segments 38 at intersections 39, as shown in FIG. 1, to form the braided pattern.

Alternatively, the body region 30, the first flanged region 40 and/or the second flanged region 50 may comprise shapes other than braided patterns. For example, one or more of these regions may comprise diamond-shaped struts, zig-zag shaped struts, or other shapes that may vary depending on the needs of the procedure.

Moreover, the stent 20 may be designed to be either balloon-expandable or self-expandable. The body region 30, the first flanged region 40 and the second flanged region 50 may be made from numerous metals and alloys, including stainless steel, nitinol, cobalt-chrome alloys, amorphous metals, tantalum, platinum, gold and titanium. The stent may also be made from non-metallic materials, such as thermoplastics and other polymers. The structure of stent 20 may also be formed in a variety of ways to provide a suitable intraluminal support structure, and may be made from a woven wire structure, a laser-cut cannula, individual interconnected rings, or any other type of stent structure that is known in the art. Optionally, one or more regions of the stent 20 may comprise a coating designed to achieve a desired biological effect.

Further, it will be apparent that while the stent 20 has been described primarily with respect to treatment of a stricture within a duct or vessel, the present embodiments may be used in other applications. For example, the apparatus and methods may be used in the treatment of aneurysms, whereby the stent 20 is coupled to a graft material along its length to provide a conduit for flow across the aneurysm, wherein the identifiable markers 69 and 79 of the proximal and distal junctions 60 and 70, respectively, identify proper placement of the stent 20 such that the first and second flanged regions 40 and 50 engage healthy tissue on opposing sides of the aneurysm.

While various embodiments of the invention have been described, it will be apparent to those of ordinary skill in the art that many more embodiments and implementations are possible within the scope of the invention. Accordingly, the invention is not to be restricted except in light of the attached claims and their equivalents. Moreover, the advantages described herein are not necessarily the only advantages of the invention and it is not necessarily expected that every embodiment of the invention will achieve all of the advantages described.

I claim:

1. A stent for use in a medical procedure, the stent comprising:
a first flanged region having proximal and distal ends, and further having a first diameter when the stent is in an expanded deployed state;
a body region having proximal and distal ends, and having a second diameter when the stent is in the expanded deployed state, wherein the first diameter of the first flanged region is greater than the second diameter of the body region; and
a proximal junction formed between the first flanged region and the body region, the proximal junction comprising at least one strut extending from the distal end of the first flanged region in a distal direction towards the proximal end of the body region,
wherein a strut at the proximal end of the body region is disposed around at least a portion of the strut of the proximal junction, and
wherein overlap between the strut at the proximal end of the body region with the strut of the proximal junction causes an increased radiopaque effect at the proximal junction.

2. The stent of claim 1 wherein the strut of the proximal junction comprises first and second segments that extend distally from the first flanged region and converge at an apex, wherein the strut at the proximal end of the body region is disposed around the apex.

3. The stent of claim 2 wherein at least one loop member encircles a zone where the strut at the proximal end of the body region is disposed around the apex.

4. The stent of claim 1 wherein the strut of the proximal junction comprises first and second segments that extend distally from the first flanged region and converge at an apex, wherein the first and second segments form an integral loop member at the apex, where the strut at the proximal end of the body region is disposed through the integral loop member.

5. The stent of claim 1 wherein the strut of the proximal junction comprises first and second segments that extend distally from the first flanged region and converge at an apex, wherein an external loop member is coupled to the strut of the proximal junction at the apex, and wherein the strut at the proximal end of the body region is disposed through the external loop member.

6. The stent of claim 1 wherein the strut of the proximal junction comprises first and second segments that extend distally from the first flanged region and converge at an apex, wherein the apex is folded over in a proximal direction to form a generally W-shape in the strut of the proximal junction, and wherein the strut at the proximal end of the body region is disposed around the W-shape formed in the strut of the proximal junction.

7. The stent of claim 6 wherein the strut at the proximal end of the body region is disposed in front of the W-shape formed in the strut of the proximal junction at least two times, and is disposed behind the W-shape formed in the strut of the proximal junction at least two times.

8. The stent of claim 1, further comprising:
a second flanged region having proximal and distal ends, and further having a diameter substantially identical to the first diameter when the stent is in the expanded deployed state; and
a distal junction formed between the second flanged region and the body region, the distal junction comprising at least one strut extending from the proximal end of the second flanged region in a proximal direction towards the distal end of the body region,
wherein a strut at the distal end of the body region is disposed around at least a portion of the strut of the distal junction, and
wherein overlap between the strut at the distal end of the body region with the strut of the distal junction causes an increased radiopaque effect at the distal junction.

9. A method for enhanced visualization of at least a portion of a stent during a medical procedure, the method comprising:
providing a stent comprising a first flanged region having proximal and distal ends, and a body region having proximal and distal ends, wherein a first diameter of the first flanged region is greater than a second diameter of the body region when the stent is in an expanded deployed state;
providing a proximal junction between the first flanged region and the body region, the proximal junction comprising at least one strut extending from the distal end of the first flanged region in a distal direction towards the proximal end of the body region; and
disposing a strut at the proximal end of the body region around at least a portion of the strut of the proximal junction,
wherein overlap between the strut at the proximal end of the body region with the strut of the proximal junction causes an increased radiopaque effect at the proximal junction.

10. The method of claim 9 wherein the strut of the proximal junction comprises first and second segments that extend distally from the first flanged region and converge at an apex, wherein the strut at the proximal end of the body region is disposed around the apex.

11. The method of claim 10 wherein at least one loop member encircles a zone in which the strut at the proximal end of the body region is disposed around the apex.

12. The method of claim 9 wherein the strut of the proximal junction comprises first and second segments that extend distally from the first flanged region and converge at an apex, wherein the first and second segments form an integral loop member at the apex, where the strut at the proximal end of the body region is disposed through the integral loop member.

13. The method of claim 9 wherein the strut of the proximal junction comprises first and second segments that extend distally from the first flanged region and converge at an apex, wherein an external loop member is coupled to the strut of the proximal junction at the apex, and wherein the strut at the proximal end of the body region is disposed through the external loop member.

14. The method of claim 9 wherein the strut of the proximal junction comprises first and second segments that extend distally from the first flanged region and converge at an apex, wherein the apex is folded over in a proximal direction to form a generally W-shape in the strut of the proximal junction, where the strut at the proximal end of the body region is disposed around the W-shape formed in the strut of the proximal junction.

15. The method of claim 14 wherein the strut at the proximal end of the body region is disposed in front of the W-shape formed in the strut of the proximal junction at least two times, and is disposed behind the W-shape formed in the strut of the proximal junction at least two times.

16. A stent for use in a medical procedure, the stent comprising:
a first flanged region having proximal and distal ends, and further having a first diameter when the stent is in an expanded deployed state;
a body region having proximal and distal ends, and having a second diameter when the stent is in the expanded deployed state, wherein the first diameter of the first flanged region is greater than the second diameter of the body region;
a proximal junction formed between the first flanged region and the body region, the proximal junction comprising at least one strut extending from the distal end of the first flanged region in a distal direction towards the proximal end of the body region; and
at least one loop member disposed along the proximal junction for facilitating coupling of the first flanged region to the body region, wherein the at least the loop member causes an increased radiopaque effect at the proximal junction.

17. The stent of claim 16 wherein a strut at the proximal end of the body region is disposed around at least a portion of the strut of the proximal junction.

18. The stent of claim 17 wherein the at least one loop member encircles a zone of overlap between the strut at the proximal end of the body region with the strut of the proximal junction.

19. The stent of claim 16 wherein the strut of the proximal junction comprises first and second segments that extend distally from the first flanged region and converge at an apex, wherein the first and second segments integrally form the at least one loop member at the apex, wherein the strut at the proximal end of the body region is disposed through the loop member.

20. The stent of claim 16 wherein the strut of the proximal junction comprises first and second segments that extend distally from the first flanged region and converge at an apex, wherein the at least one loop member is an external loop member that is coupled to the strut of the proximal junction at the apex, and wherein the strut at the proximal end of the body region is disposed through the external loop member.
